(19) United States
(12) Patent Application Publication
(10) Pub. No.: US 2010/0266501 A1
(43) Pub. Date: Oct. 21, 2010

(54) METHODS AND COMPOSITIONS FOR ORGAN PROTECTION

(75) Inventors: Markus Meyer, Randolph, VT (US); Martin M. LeWinter, Williston, VT (US); Harold L. Dauerman, Shelburne, VT (US); Stephen P. Bell, Milton, VT (US)

Correspondence Address: WOLF GREENFIELD & SACKS, P.C., 600 ATLANTIC AVENUE, BOSTON, MA, 02210-2206, US

(73) Assignee: University Of Vermont and State Agricltural College, Burlington, VT (US)

(21) Appl. No.: 12/312,093

(22) PCT Filed: Nov. 6, 2007
(86) PCT No.: PCT/US07/23368

§ 371 Date: Mar. 2, 2010

Related U.S. Application Data
(60) Provisional application No. 60/857,434, filed on Nov. 7, 2006.

(51) Int. Cl. A61K 49/00; A61P 9/10; A61P 9/14; A61P 13/12; A61P 39/06

(52) U.S. Cl. 424/9.1

(57) ABSTRACT

The invention relates in some aspects to compositions that include a contrast agent and an antioxidant compound and the use of such compositions to prevent (e.g., reduce) organ damage in a subject. In some aspects, the invention relates to compositions that include N— acetylcysteine (NAC) and a contrast agent and the use of such compositions for preventing organ damage in a subject. Methods of preventing organ damage may include the simultaneous administration of a composition of the invention, such that a contrast agent and an antioxidant compound or a contrast agent and NAC are administered intravascularly to a subject. Examples of organ damage that may be treated with compositions and methods of the invention include contrast-induced and non-contrast induced organ damage associated with myocardial ischemia, myocardial infarction, reperfusion damage, nephropathy, etc. The invention, in some aspects, also includes kits that may include a contrast agent and NAC and/or an antioxidant compound.

### RELATED APPLICATIONS

This application claims the benefit under 35 U.S.C. §119(e) of U.S. provisional application Ser. No. 60/857,434, filed Nov. 7, 2006, the disclosure of which is incorporated by reference herein in its entirety.

### FIELD OF THE INVENTION

The invention relates in some aspects to compositions that include a contrast agent and an antioxidant compound and the use of such compositions to prevent organ damage in a subject. In some aspects, the invention relates to compositions that include N-acetylcysteine (NAC) and a contrast agent and the use of such compositions to prevent organ damage in a subject. Examples of organ damage that may be prevented using compositions and methods of the invention may include organ damage associated with myocardial ischemia, myocardial infarction, myocardial reperfusion damage, nephropathy, etc. The invention also includes kits that may include contrast agent and NAC and/or another antioxidant compound.

### BACKGROUND OF THE INVENTION

Contrast induced nephropathy (CIN) remains a significant complication after cardiac catheterization. While the overall risk of CIN complicating cardiovascular procedures is now less than 5 percent, this risk can exceed 30 percent in the setting of preexistent renal failure, diabetes mellitus, reduced left ventricular ejection fraction and larger contrast loads (1-3). Because CIN is associated with increased mortality, recent efforts have focused on finding adjunctive agents that reduce the nephrotoxic effects of the available contrast agents (4). N-Acetylcysteine (NAC) is an antioxidant agent with potential myocardial and renal protective effects (5-7). Although the use of NAC for prevention of CIN is still controversial, two recent meta-analyses concluded that the majority of clinical trials have demonstrated beneficial effects (8, 9). In a recent trial of combined oral and intravenous risk-adjusted dose administration, an enhanced benefit of high dose NAC was demonstrated among patients undergoing percutaneous coronary interventions (10).

### SUMMARY OF THE INVENTION

The invention is based on the administration to a subject of a mixture of a contrast agent with N-acetylcysteine (NAC) or a mixture of a contrast agent with an antioxidant agent can prevent organ damage in a subject. Administration of a mixture of a contrast agent with NAC or another antioxidant agent can reduce organ damage and subject mortality. The invention relates, in part, to methods, compositions, and kits for preventing organ damage in a subject undergoing administration of a contrast agent.

According to one aspect of the invention, compositions that include a contrast agent and N-acetylcysteine (NAC) are provided. In some embodiments, the NAC is crosslinked to the contrast agent. In certain embodiments, the amount of NAC in the composition is about 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, or more of NAC per mL (cc) of contrast agent. In some embodiments, the contrast agent is an ionic contrast agent. In certain embodiments, the contrast agent is a non-ionic contrast agent. In some embodiments, the composition is formulated for intra-arterial injection to a subject. In some embodiments, the composition is formulated for intravenous injection in to a subject.

According to another aspect of the invention, methods for administering a contrast agent to a subject are provided. The methods include administering the composition of claim A1 to the subject, wherein the contrast agent and N-acetylcysteine (NAC) of the composition are simultaneously administered to the subject. In certain embodiments, the NAC is crosslinked to the contrast agent. In some embodiments, the amount of NAC administered in the composition is an amount effective to prevent organ damage in the subject. In some embodiments, the organ damage is myocardial ischemia, myocardial infarction, and/or reperfusion damage. In some embodiments, the organ damage is renal damage. In certain embodiments, the renal damage is nephropathy. In some embodiments, the nephropathy is contrast-induced nephropathy (CIN). In certain embodiments, the organ damage is brain damage. In some embodiments, the organ damage is contrast-induced organ damage. In some embodiments, the organ damage is not contrast-associated organ damage. In certain embodiments, the subject is a mammal. In some embodiments, the subject is a human. In some embodiments, administration is intra-arterial administration. In certain embodiments, administration is intravenous administration. In some embodiments, the contrast agent is an ionic contrast agent. In some embodiments, the contrast agent is a non-ionic contrast agent. In certain embodiments, the amount of NAC administered to the subject is about 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, or more of NAC per mL (cc) of contrast agent administered to the subject.

According to yet another aspect of the invention, compositions that include a contrast agent and an antioxidant compound are provided. In some embodiments, the antioxidant compound is N-acetylcysteine (NAC). In some embodiments, the antioxidant compound is crosslinked to the contrast agent. In certain embodiments, the antioxidant compound is sodium thiosulfate (STS), GSH ethyl ester, D-methionine, thiol amifostine (Ethyol, WR2721), Vitamin E, tocopherol, dithiotreitol, mercaptoethanol, or glutathion. In some embodiments, the amount of the antioxidant compound in the composition is from about 0.1 mg to at least about 200 mg or more of antioxidant per mL (cc) of contrast agent. In certain embodiments, the amount of NAC in the composition is about 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, or more of NAC per mL (cc) of contrast agent. In some embodiments, the contrast agent is an ionic contrast agent. In some embodiments, the contrast agent is a non-ionic contrast agent. In certain embodiments, the composition is formulated for intra-arterial injection to a subject. In some embodiments, the composition is formulated for intravenous injection into a subject.

According to yet another aspect of the invention, methods for administering a contrast agent to a subject are provided. The methods include administering any of the compositions of any embodiments of the foregoing aspects of the invention to the subject, wherein the contrast agent and the antioxidant compound of the composition are simultaneously administered to the subject. In some embodiments, the antioxidant compound is crosslinked to the contrast agent. In certain embodiments, the amount of antioxidant compound administered in the composition is an amount effective to prevent organ damage in the subject. In some embodiments, the organ damage is myocardial ischemia, myocardial infarction, and/or reperfusion damage. In certain embodiments, the organ damage is renal damage. In some embodiments, the renal damage is nephropathy. In some embodiments, the nephropathy is contrast-induced nephropathy (CIN). In some embodiments, the organ damage is brain damage. In certain embodiments, the organ damage is contrast-induced organ damage. In some embodiments, the organ damage is not contrast-associated organ damage. In certain embodiments, the subject is a mammal. In some embodiments, the subject is a human. In some embodiments, administration is intra-arterial administration. In certain embodiments, administration is intravenous administration. In some embodiments, the contrast agent is an ionic contrast agent. In some embodiments, the contrast agent is a non-ionic contrast agent. In some embodiments, the amount of antioxidant compound administered to the subject is from about 0.1 mg to 200 mg or more of antioxidant compound per mL (cc) of contrast agent administered to the subject. In certain embodiments, the antioxidant compound is NAC. In some embodiments, the amount of NAC compound administered to the subject is from about 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, or more of NAC per mL (cc) of contrast agent administered to the subject.

According to yet another aspect of the invention, kits are provided. The kits include a contrast agent and N-acetylcysteine (NAC), and optionally include directions for crosslinking the NAC to the contrast agent and/or directions for administering the contrast agent and the NAC to a subject. In some embodiments, the NAC is crosslinked to the contrast agent. In certain embodiments, the contrast agent and the NAC are in separate containers. In some embodiments, the contrast agent and the NAC are in a same solution in a same container. In some embodiments, the contrast agent and the NAC are formulated for intra-arterial administration. In certain embodiments, the contrast agent and the NAC are formulated for intravenous administration. In some embodiments, the contrast agent is an ionic contrast agent. In some embodiments, the contrast agent is a non-ionic contrast agent. In certain embodiments, the kit also includes one or more containers containing one or more reagents for detecting the presence of organ damage in the subject. In some embodiments, the organ damage is myocardial ischemia, myocardial infarction, and/or reperfusion damage. In some embodiments, the organ damage is nephropathy. In some embodiments, the nephropathy is contrast-induced nephropathy (CIN). In certain embodiments, the organ damage is brain damage. In some embodiments, the one or more reagents are reagents for detecting presence and/or a level of a marker compound in a sample obtained from the subject. In some embodiments, the marker compound is KIM-1, cystatin C, creatinine, or urea levels in a sample obtained from the subject. In certain embodiments, the sample is a urine or blood sample from the subject.

According to yet another aspect of the invention, kits are provided. The kits include a contrast agent and an antioxidant compound, and optionally include directions for crosslinking the antioxidant compound to the contrast agent and/or directions for administering the contrast agent and the antioxidant compound to a subject. In some embodiments, the antioxidant compound is crosslinked to the contrast agent. In certain embodiments, the contrast agent and the antioxidant compound are in separate containers. In some embodiments, the contrast agent and the antioxidant compound are in a same solution in a same container. In some embodiments, the contrast agent and the antioxidant compound are formulated for intra-arterial administration. In certain embodiments, the contrast agent and the antioxidant compound are formulated for intravenous administration. In some embodiments, the contrast agent is an ionic contrast agent. In certain embodiments, the contrast agent is a non-ionic contrast agent. In some embodiments, the kit also includes one or more containers containing one or more reagents for detecting the presence of organ damage in the subject. In some embodiments, the organ damage is myocardial ischemia, myocardial infarction, and/or reperfusion damage. In certain embodiments, the organ damage is nephropathy. In some embodiments, the nephropathy is contrast-induced nephropathy (CIN). In some embodiments, the organ damage is brain damage. In certain embodiments, the one or more reagents are reagents for detecting presence and/or a level of a marker compound in a sample obtained from the subject. In some embodiments, the marker compound is KIM-1, cystatin C, creatinine, or urea levels in a sample obtained from the subject. In some embodiments, the sample is a urine or blood sample from the subject.

### BRIEF DESCRIPTION OF THE DRAWINGS

FIG. 1 shows two bar graphs illustrating the area at risk and areas of infarction in various studies. FIG. 1A depicts the area at risk and the area of infarction in five control (Iopamidol contrast-only) and five N-Acetylcysteine (Iopamidol contrast plus 11.3 mg/mL N-Acetylcysteine) treated pig hearts expressed as percentage of ventricular myocardium. FIG. 1B depicts the percentage of the area at risk that was infarcted in both control and N-Acetylcysteine treated animals.

FIG. 2 shows a diagram of a ventricular slice and a bar graph showing results of infarct studies. FIG. 2A demonstrates an example of the infarcted area (black) in a left ventricular slice obtained from a control (Iopamidol contrast-only) and N-Acetylcysteine (Iopamidol contrast with 11.3 mg/mL N-Acetylcysteine) treated animal. The infarction in the NAC treated animal is mottled with interspersed areas of viable myocardium (gray). The numerical values reflect the ratio of the infarct circumference to infarct area an index for infarct heterogeneity. The bar graph in FIG. 2B depicts the results of the group analysis.

FIG. 3 is a bar graph depicting the percentages of TUNEL positive nuclei reflecting apoptosis in non-ischemic, area-at-risk and in infarcted myocardium from control and N-Acetylcysteine treated animals.

FIG. 4 shows a bar graph demonstrating creatinine concentrations prior to (pre-contrast) and one day after (post-contrast) administration of intracoronary Iopamidol contrast agent (200 ml). Where indicated N-Acetylcysteine (11.3 mg/mL) was added to the contrast agent.

### DETAILED DESCRIPTION OF THE INVENTION

The invention relates, in part, to compositions that include a contrast agent and an organ-protection agent such as NAC or another antioxidant. The invention also relates, to the methods of administering such compositions to reduce organ damage in subjects. The simultaneous delivery of a contrast agent in solution with an organ-protection agent provides an effective method of protecting organs from damage, including ischemic damage, reperfusion damage, contrast-associated damage, etc.

As used herein the term “contrast-protectant composition” means a composition that comprises a contrast agent and NAC and also means a composition that comprises a contrast agent and an antioxidant. A contrast-protectant composition of the invention is also referred to herein as “protective contrast-NAC”, or PC-NAC. It will be understood that a composition of the invention may comprise more than one antioxidant and/or may comprise NAC and one or more other antioxidant compounds.

NAC is a derivative of the amino acid L-cysteine and is a precursor in the formation of the antioxidant glutathione in the body. The thiol (sulfhydryl) group of NAC confers antioxidant effects and is able to reduce the production oxygen-derived free radicals. NAC is primarily used as a mycolytic and in the management of paracetamol overdose. An intravenous form of NAC is available both in Europe (Fluimucyl®) and in the United States (Acetadote®) where it is approved to treat Acetaminophen (Tylenol®) overdoses. Oral administration of NAC has also been suggested for the reduction of contrast-induced nephropathy (CIN). Although NAC has antioxidant properties, its ability to prevent organ damage may be associated with or caused entirely or in part by other properties.

An antioxidant compound is a compound that reduces oxidative damage (damage due to oxygen) such as that caused by oxygen-derived free radicals. Free radicals are highly reactive chemicals that attack molecules by capturing electrons and thus modifying chemical structures. Antioxidant compounds that may be useful in compositions and methods of the invention include, but are not limited to, sodium thiosulfate (STS), GSH ethyl ester, D-methionine, thiol amifostine (Ethyol, WR2721), Vitamin C and E, tocopherol, dithiotreitol, mercaptoethanol and glutathion.

Contrast agents, which are also referred to herein as “contrast” are compounds that are administered to a subject to enhance visualization of internal structures of the body. Contrast is used in many types of imaging procedures such as ultrasound, magnetic resonance imaging (MRI), computerized Axial Tomography (CAT) imaging, and X-ray imaging. Contrast used to during the course of imaging procedures that utilize X-ray beams may be referred to as radiographic contrast. Intravenous or intrarterial contrast is used for diagnostic and therapeutic procedures and is useful to enhance imaging of blood vessels and to image the structure of organs like the brain, spine, liver, kidney, or the peripheral vasculature in the extremities. Contrast injected into the bloodstream of a subject circulates throughout the body, permitting enhanced visualization of blood vessels and organs that “take up” the contrast. Radiographic and other contrast agents injected into the heart allow visualization of heart vessels and structure in order to identify the location of coronary artery blockages and to delineate the function and size of the heart chambers.

A contrast agent used in a composition and/or method of the invention may be an ionic contrast agent or may be a non-ionic contrast agent. There are numerous commercially available ionic and non-ionic contrast agents that may be used in compositions and methods of the invention.

Non-limiting examples of ionic contrast agent suitable for use in compositions and methods of the invention include Renovue®-DIP (iodamide meglumine) (Squibb), Conray® 30 (iothalamate meglumine) (Mallinckrodt), Hypaque® Meglumine 30% (diatrizoate meglumine) (Winthrop), Reno-M-DIP® (amidotrizoate meglumine; diatrizoate meglumine) (Squibb), Urovist® Meglumine DIU/CT (diatrizoate meglumine) (Berlex), Hypaque® Sodium 25% (diatrizoate sodium) (Winthrop), Conray® 43 (iothalamate meglumine) (Mallinckrodt), Angiovist® 282 (diatrizoate meglumine) (Berlex), Hypaque® Meglumine 60% (diatrizoate meglumine) (Winthrop), Reno-M-60® (diatrizoate meglumine) (Squibb), Conray® (iothalamate meglumine) (Mallinckrodt), Angiovist® 292 (diatrizoate meglumine sodium) (Berlex), MD-60® (diatrizoate meglumine sodium) (Mallinckrodt), Renografin®-60 (diatrizoate meglumine sodium) (Squibb), Hypaque® Sodium 50% (diatrizoate sodium) (Winthrop), Renovue®-65 (iodamide meglumine) (Squibb), Urovist® Sodium 300 (diatrizoate sodium) (Berlex), Renovist® II (diatrizoate meglumine sodium) (Squibb), Hexabrix (ioxaglate meglumine sodium) (Mallinckrodt), Conray® 325 (iothalamate sodium) (Mallinckrodt), Diatrizoate Meglumine 76% (diatrizoate meglumine) (Squibb), Angiovist® 370 (diatrizoate meglumine sodium) (Berlex), Hypaque®-76 (diatrizoate meglumine sodium) (Winthrop), MD-76® (diatrizoate meglumine sodium) (Mallinckrodt), Renografin®-76 (diatrizoate meglumine sodium) (Squibb), Renovist® (diatrizoate meglumine sodium) (Squibb), Hypaque®-M, 75% (diatrizoate meglumine sodium) (Winthrop), Conray® 400 (iothalamate sodium) (Mallinckrodt), Vascoray® (iothalamate meglumine sodium) (Mallinckrodt), Hypaque®-M, 90% (diatrizoate meglumine sodium) (Winthrop), Angio Conray® (iothalamate sodium) (Mallinckrodt), and ioxithalamate.

Non-limiting examples of non-ionic contrast agents suitable for use in compositions and methods of the invention include: Isovue®-128 (Squibb) (iopamidol 26.1%), Omnipaque® 140 (Winthrop) (iohexyl 30.2%), Optiray® 160 (Mallinckrodt) (ioversol 33.9%), Isovue®-200 (Squibb) (iopamidol 40.8%), Omnipaque® 240 (Winthrop) (iohexyl 51.8%), Optiray® 240 (Mallinckrodt) (ioversol 50.9%), Isovue®-300 (Squibb) (iopamidol 61.2%), Omnipaque®-300 (Winthrop) (iohexyl 64.7%), Optiray® 320 (Mallinckrodt) (ioversol 67.8%), Omnipaque® 350 (iohexyl 75.5%) (Winthrop), Isovue®-370 (Squibb) (iopamidol 75.5%), iodixanol, and iomeprol.

In some aspects of the invention, a contrast agent mixed with (e.g., in solution with) NAC and/or an antioxidant is administered to a subject. Unlike previous methods of administering NAC and contrast agents, wherein NAC and contrast were administered separately to a subject, methods of the invention include the administration of NAC and a contrast agent or administration of an antioxidant and a contrast agent that are in a common solution and may be simultaneously delivered to a subject. As used herein the term “common” when used in reference to a composition or solution means “the same”. As used herein, the term “simultaneous” with respect to delivery of contrast and a protectant compound means administration of a contrast agent and a protectant compound in a common composition. Thus, using methods of the invention there is less spatial and/or temporal separation between delivery of a protectant compound and delivery of contrast than occurs when contrast and a protectant compound are administered using previous methods. Combining a protectant compound and a contrast agent in a common solution prior to administration permits simultaneous administration of a protectant compound and a contrast agent to the subject at a common administration site. For example, a contrast-protectant composition may be administered to a subject via an intra-arterial catheter.

Administration of contrast and a protectant compound together in a common composition via a common administration means (e.g., a catheter) permits simultaneous contact of the contrast and the protectant compound with tissues of the subject. For example, in a cardiac catheterization procedure of the invention, an intra-arterial catheter may be placed in a blood vessel and into the heart and a contrast-protectant composition may be injected through the catheter into the heart. The contrast in the administered contrast-protectant composition provides information on the physical state of the coronary arteries (e.g., to narrowing or blockage) and allows a functional assessment of the heart valves, chambers, and blood vessels via imaging. The protectant compound in the administered contrast-protectant composition prevents organ damage in the subject. For example, a composition of the invention may prevent damage in vessels of the heart and in the heart itself, as well as in other organs. Because the administered contrast-protectant composition includes both a contrast agent and a protectant compound, the subject's vessels and heart are simultaneously contacted with contrast agent and the administered protectant compound. In a subject receiving contrast that is not administered as part of a common composition with a protectant, the subject's vessels and organs may be contacted with contrast in the absence of an administered protectant compound. The simultaneous contact of a subject's tissues with contrast and a protectant compound, as occurs in the methods of the invention, may be more effective in preventing organ damage than alternative methods of administering contrast.

The contrast-protectant compositions of the invention may prevent (e.g., reduce) organ damage in a subject in whom they are administered. Organ damage may be a result of numerous factors in the subject including, but not limited to, reperfusion, contrast toxicity, etc. Types of organ damage that may be prevented by compositions and/or methods of the invention include, but are not limited to: myocardial ischemia, myocardial infarction, myocardial stunning, infarct propagation, reperfusion damage, renal damage, nephropathy, contrast-induced nephropathy, brain damage, liver damage, cell death, etc. Organ damage prevented by compositions and/or methods of the invention may be contrast-induced organ damage or may be organ damage that is not associated with the administration of contrast (e.g., non-contrast-induced organ damage). In certain embodiments, organ damage prevented by compositions and/or methods of the invention includes both contrast-induced organ damage and non-contrast-induced organ damage in the subject.

A contrast-protectant composition of the invention may be used to prevent organ damage in subjects undergoing a contrast procedure. Compositions and methods of the invention may be used prophyactically to treat a subject who is at risk of organ damage. A subject at risk for organ damage may be a subject undergoing a contrast administration and/or a subject with heart failure, myocardial ischemia, reperfusion, etc.

As used herein, with respect to compositions, methods, and kits of the invention, a subject is a human, non-human primate, cow, horse, pig, sheep, goat, dog, cat, rodent, or any other suitable mammalian or non-mammalian animal. In all embodiments, human subjects are preferred. Particularly important subjects to which the present invention can be applied are subjects to whom a contrast agent is to be administered. The term “subject to whom a contrast agent is to be administered” as used herein, means an individual who is to be administered a contrast agent for a diagnostic and/or therapeutic procedure (e.g., a subject in need of administration of a contrast agent). Compositions of the invention may be used to administer contrast to a subject in need of contrast agent administration. A composition comprising a contrast agent and a protectant compound of the invention may be administered to a subject for diagnostic and/or therapeutic procedures. Methods and compositions of the invention may be used in subjects for diagnostic and therapeutic imaging procedures that include the administration of contrast.

In some aspects of the invention, a composition comprising a contrast agent and NAC or a composition comprising a contrast agent and an antioxidant may be administered to a subject as part of a cardiac procedure (e.g., a cardiac catheterization). As used herein the term “cardiac procedure,” refers to any technique involving the heart and/or a blood vessel (artery, vein) associated with the heart. Examples of cardiac procedures may include, but are not limited to, angiograms and venograms, including those performed in conjunction with therapeutic procedures such as coronary angioplasty and stent placement. In some embodiments of the invention, injection of a contrast-protectant composition may be done as part of a non-cardiac procedure. As used herein a “non-cardiac procedure” means a technique that involves an organ (e.g., brain, kidney, etc.), region of the body (e.g., thorax, head, pelvis), or blood vessel that is not of a body region that would make it a cardiac procedure. Examples of non-cardiac procedures may include, but are not limited to, angiograms (e.g., to a non-cardiac region of the body such as the head, pelvis, lungs, etc.) or a venogram (e.g., to a non-cardiac vessel such as a femoral blood vessel, a cerebral blood vessel, etc.)

Administration of a contrast-protectant composition of the invention may prevent organ damage in a subject. As used herein the term “prevent” means to reduce the occurrence of organ damage that would have occurred in the subject without the administration of the composition to the subject. As used herein prevention of organ damage may be a prophylactic treatment of organ damage. Thus, a subject may be treated prophylactically using compositions and methods of the invention to prevent organ damage. Prevention of organ damage in a subject may mean the that 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or up to 99% (including all percentages between) of organ damage that would have occurred in the subject without the administration of the contrast-protectant composition occurs in the subject administered the contrast-protectant composition. Thus, administration of contrast with NAC or an antioxidant need not eliminate 100% of organ damage in a subject to be considered to be effective at preventing organ damage, but rather reduces the amount of organ damage by a statistically relevant amount compared to an amount expected in a control subject not treated with a contrast-protectant composition of the invention.

In general, contrast agents can be administered to subjects using a number of methods, including administration by parenteral injection (e.g., intravenous, intra-arterial, intra-thecal, intra-abdominal, subcutaneous, intramuscular), oral delivery (e.g., as a tablet or a drink) or as an enema. In preferred embodiments of the invention, administration of a composition comprising a contrast agent and NAC and/or a contrast agent and an antioxidant is via intravenous or intra-arterial administration. In some embodiments of the invention, a composition comprising a contrast agent and NAC or a composition comprising a contrast agent and an antioxidant may be administered via a catheter placed into a vessel, such as an intra-arterial catheter.

An effect of administration of a contrast-protectant composition of the invention on the prevention of organ damage in a subject may be determined based on a comparison of the effects of such administration with controls according to the invention. A control may be a predetermined value, which can take a variety of forms. It can be a measure of established risk of a procedure. A control may also be a single cut-off value, such as a median or mean. It can be established based upon comparative groups, such as in groups having contrast administered without administration with NAC or other antioxidant, or a group having contrast and NAC and/or an antioxidant administered separately, e.g., not administered within a common composition. Another example of comparative groups may be groups having myocardial ischemia or ischemic symptoms and groups without ischemia or ischemic symptoms and/or groups with other physiological disease or damage such as vascular disease, heart disease, kidney disease, etc. Another comparative group may be a group or subjects with a family history of organ damage upon catheterization or a family history of renal disease, heart disease, stroke, etc. and a group without such a family history. A predetermined value can be arranged, for example, where a tested population is divided equally (or unequally) into groups, such as a low-risk group, a medium-risk group and a high-risk group or into quadrants or quintiles, the lowest quadrant or quintile being individuals with the lowest risk of organ damage and the highest quadrant or quintile being individuals with the highest risk of organ damage.

A predetermined value, of course, will depend upon the particular population selected. For example, an apparently healthy population will have a different ‘normal’ range than will a population that is known to have a condition related to vascular disease, cardiac disease, diabetes, renal disease, etc. Accordingly, the predetermined value selected may take into account the category in which an individual falls. Appropriate ranges and categories can be selected with no more than routine experimentation by those of ordinary skill in the art. Thus, prevention of organ damage provided by administering a contrast-protectant composition of the invention may be assessed by comparison to a selected control.

Methods of the invention may include administration of a contrast-protectant composition to a subject in an effective amount. An effective amount of a contrast-protectant composition is that amount of protectant effective to reduce the level of organ damage in the subject (e.g., prevent some or all organ damage in the subject) and that amount of contrast sufficient to permit imaging as medically indicated for the subject. Thus, compositions of the invention may be administered in amounts effective to prevent and/or treat organ damage and to permit imaging. Typically an effective amount of a compound that decreases organ damage will be determined in clinical trials, establishing an effective dose for a test population versus a control population in a blind study. In some embodiments, an effective amount will be an amount that results in a desired response, e.g., an amount that diminishes or eliminates organ damage in a subject undergoing imaging. Thus, an effective amount may be the amount that when administered reduces the amount of organ damage from the amount of organ damage that would occur in the subject or tissue without the administration of the contrast-protectant composition of the invention. In the case of treating a subject the desired response is inhibiting as much of the organ damage as possible up to 100% inhibition. This may involve only reducing some organ damage, although more preferably, it involves preventing most or all of the organ damage in the subject. This can be monitored by routine organ damage diagnostic methods known to one of ordinary skill in the art.

Effective amounts of a contrast-protectant composition of the invention may also be determined by assessing physiological effects in a subject that result from administration of a contrast-protectant composition of the invention. One example of a physiological effect may be a delay or reduction in the onset of organ damage symptoms following administration of a contrast-protectant composition of the invention. Assays that can be used to detect organ damage include, but are not limited to assays to detect a level or presence of a marker compound in a subject that has been treated with a composition of the invention. As used herein the term “marker compound” is used to denote a compound that may be present in normal tissues and subjects but that has either an elevation or reduction of the normal level in a subject with organ damage. Thus, a change in the level of a marker compound can be indicative of organ damage in a subject. The level of one or more marker compounds can be monitored in a subject and the detected levels may be compared to control levels (e.g., normal levels or levels in the subject prior to administration of a composition of the invention, etc.). Examples of such marker compounds that may be measured as an indication of organ damage, though not intended to be limiting are KIM-1, Cystatin C, creatinine, and/or urea. In some embodiments, a level of one or more marker compounds can be determined in a bodily fluid or tissue from a subject, including, but not limited to a blood, urine, and/or a tissue sample from the subject. The level of a marker compound may be compared to a control level of the marker compound as a measure of whether or not organ damage is present in the subject. This may be ascertained by detecting levels of one or more marker compounds in a subject or sample from a subject to whom a contrast-protectant composition of the invention was administered. Those of ordinary skill in the art will also recognize addition ways to monitor a subject for organ damage and for measuring the level of the response to administration of a composition of the invention.

As used herein a contrast-protectant composition may be prepared such that the mixture includes a predetermined ratio of contrast agent to protectant compound, such that an effective amount of the agent and an effective amount of the compound may be administered simultaneously to a subject. As described herein, the amount of a contrast agent administered to a subject is determined based on standard contrast dosing methodologies that are known to those of skill in the art. A dose of a protectant compound administered to a subject may be relative to the dose of a contrast agent administered to the subject, such that a subject who is administered a higher dose of contrast will concomitantly be administered a higher dose of a protectant compound. A composition of the invention that comprises NAC and a contrast agent may include an amount of NAC that is about 0.1 mg, 0.2 mg., 0.3 mg. 0.4 mg, 0.5 mg, 0.6 mg., 0.7 mg, 0.8 mg, 0.9 mg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 300 mg, 400 mg, 500 mg, or more NAC to per cc (ml) of contrast agent, including all values in between each of the amounts of NAC.

A composition of the invention that includes a contrast agent and an antioxidant may include an amount of an antioxidant that is about 0.1 mg, 0.2 mg, 0.3 mg, 0.4 mg, 0.5 mg, 0.6 mg, 0.7 mg, 0.8 mg, 0.9 mg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 300 mg, 400 mg, 500 mg, 600 mg, 700 mg, 800, mg, 900 mg, 1000 mg or more mg of antioxidant compound per cc (ml) of contrast agent, including all values in between each of the amounts of antioxidant.

A contrast agent may be free in solution with an NAC and/or antioxidant compound in a composition of the invention. A contrast agent may also be attached to (e.g., crosslinked to a protectant compound such as NAC and/or an antioxidant compound in a contrast-protectant composition of the invention. A contrast-protectant composition of the invention is a composition that excludes blood prior to administration to a subject.

An amount of therapeutic or diagnostic treatment of a subject may be varied by increasing or decreasing the amount of a contrast-protectant composition administered to the subject by changing the therapeutic composition administered (e.g., altering the ratio of contrast to protectant in the composition, etc.), by changing the dosage timing and so on. An amount that is an effective amount will vary with the particular type of contrast agent and the contrast administration procedure being followed, the age and physical condition of the subject being treated, the total amount of contrast to be administered to the subject, the presence of additional diseases and/or disorders in the subject (e.g., cardiac disease, coronary artery disease, diabetes, stroke, renal insufficiency, etc.), the nature of any concurrent therapy, the specific route of administration, and additional factors within the knowledge and expertise of the health practitioner. For example, an effective amount may depend upon the degree to which an individual has renal or cardiac disease or a condition that may affect the likelihood of organ damage occurring in the subject. Such factors are well known to those of ordinary skill in the art and can be addressed with no more than routine experimentation.

It is generally preferred that a maximum dose of a contrast-protectant composition of the invention (alone or in combination with other therapeutic agents) be used, that is, the highest safe dose according to sound medical judgment. It will be understood by those of ordinary skill in the art, however, that a patient may insist upon a lower dose or tolerable dose for medical reasons, psychological reasons or for virtually any other reasons. The dosage of contrast-protectant composition of the invention administered to a subject can be chosen in accordance with different parameters, in particular in accordance with the mode of administration used and the state of the subject. In the event that a response in a subject is insufficient at the initial doses applied, higher doses may be employed to the extent that patient tolerance permits.

It will be understood that the amount of the contrast-protectant composition administered to a subject will depend on the amount of contrast necessary to administer to the subject to attain the desired visualization and/or imaging using the contrast. Those of ordinary skill in the art will be able to calculate the amount of contrast for administration using standard contrast dosing parameters well known in the art. For example, in some subjects, a larger dose of contrast will be administered for successful imaging and the dose of contrast will be determined based on standard contrast-dosing parameters.

The absolute amount of a contrast-protectant composition that is administered will depend upon a variety of factors, including the material selected for administration and individual subject parameters including age, physical condition, size, weight, and the stage of the disease or condition. These factors are well known to those of ordinary skill in the art and can be addressed with no more than routine experimentation.

A contrast-protectant composition used in the foregoing methods preferably are sterile and contain an effective amount of NAC and/or an antioxidant compound that will prevent organ damage in a unit of weight or volume suitable for administration to a patient. Contrast-protectant compositions of the invention may be administered alone, in combination with each other, and/or in combination with other drug therapies, or other treatment regimens that are administered to subjects. In some embodiments a contrast-protectant composition comprises contrast, NAC, and one or more antioxidant compounds.

Administration of a contrast-protectant composition of the invention to a mammal other than a human, e.g., for testing purposes or veterinary therapeutic purposes, is carried out under substantially the same conditions as described above. It will be understood by one of ordinary skill in the art that this invention is applicable to prevent organ damage in both human and animal subjects.

Also within the scope of the invention are kits comprising one or more contrast agents and NAC and/or an antioxidant compound. In some embodiments of the invention, a kit may to comprise contrast and NAC provided in a single solution in a container. In some embodiments of the invention, a kit may comprise contrast and an antioxidant that are in a single solution in a container. A kit may also include instructions for use, including, but not limited to instructions for preparation of a contrast-protectant composition of the invention. The kits can further contain at least one additional reagent, such as one or more additional antioxidant compounds. In some embodiments, a kit may include components for detecting a level of a marker compound and may optionally include instructions for using the components to determine the presence and/or level of organ damage in a subject. A kit may also contain catheters, syringes, needles, and other components useful to carry out the methods of the invention.

The invention will be more fully understood by reference to the following examples. These examples, however, are merely intended to illustrate the embodiments of the invention and are not to be construed to limit the scope of the invention.

### EXAMPLES

### Administration of a Contrast Agent in Conjunction with N-Acetylcysteine (NAC)

The addition of NAC to an intra-coronary contrast agent has now been tested and determined to match the NAC dose to the contrast volume and to provide adjusted renal protection with a potential for myocardial protection. In tests of this approach a fixed NAC-contrast mixture was used during coronary angiography in a pig model of myocardial ischemia and reperfusion. The frequency of acute arrhythmias was analyzed and, after 24 hours, the area of myocardium at risk for infarction, infarct size, infarct morphology, apoptosis, and renal function were assessed.

### Methods

### Dose Escalation Experiments and Safety of Intracoronary NAC

Although intracoronary NAC infusions are reported to be safe in humans, the effects of intracoronary bolus injections are not known (11). Accordingly, the effects of escalating doses of intracoronary NAC (Cumberland Pharmaceuticals, Nashville, Tenn.) mixed with contrast medium on blood pressure and cardiac rhythm were tested in two healthy farm raised pigs (23 and 26 kg). All aspects of animal care were in accordance with the guidelines published by the National Institutes of Health in the “Guide for the Care and Use of Laboratory Animals” and the standards of the Institutional Animal Care and Use Committee to of the University of Vermont.

The pigs were anesthetized with Isoflurane after intramuscular pre-medication with Ketamine (20 mg/kg). After endotracheal intubation they were ventilated (Harvard Apparatus, Holliston, Mass.) with a mixture of oxygen and 1-3 percent isoflurane. A surgical level of anesthesia was confirmed prior to obtaining percutaneous femoral arterial access using a 6-French sheath. A JR 4 or HS guide catheter was inserted and placed at the ostium of the left main coronary artery. Continuous twelve-lead ECG and catheter based pressures were digitally recorded with the administration of bolus injections of NAC. The initial dose was 56.6 mg of NAC diluted in 5 mL of. Iopamidol (Isovue 370®, Bracco Pharmaceuticals, Princeton, N.J.). This dose of 11.3 mg NAC/mL is referred to as the target dose because it would deliver a dose of 1850 milligrams of NAC to an average patient undergoing a percutaneous coronary intervention. This is similar to the intravenous dose used by Marrenzi et al. and about ten-fold below the dose used for the intravenous treatment of acetaminophen intoxications (10, 12). Following at least five injections of the target dose, a total of four high dose injections were performed, two of 250 mg per 5 mL bolus and two of 500 mg NAC per 5 mL bolus (equal to 4.4× and 8.8× target dose). After the experiment, the pigs were monitored for an additional hour prior to being transported to the housing facility. Blood pressure and QT interval were analyzed at the end of three succeeding respiratory cycles (6, 12 and 18 seconds after injection) and are reported as a mean percent change from baseline.

### Safety and Efficacy of NAC in an Ischemia-Reperfusion Model

Experimental Protocol: A total of twelve pigs (27.6±4.6 kg) were assigned to either Iopamidol contrast alone (n=5) or NAC enhanced contrast using a NAC concentration of 11.3 mg/mL (n=7). The contrast dilution at this dose was six percent, which does not interfere with interpretation of coronary angiograms based on previous contrast measurements using quantitative fluoroscopy (13). Sedation, anesthesia and instrumentation of the pigs were accomplished in accordance to the dose escalation study. All pigs received a bolus of 2000 units of heparin sulfate after obtaining femoral artery access. A guide wire (PT2, 0.014″×185 cm, Boston Scientific, Natick, Mass.) was then advanced into the left anterior descending (LAD) coronary artery followed by inflation of a balloon (Monorail Maverick, 2.5×15 mm, Boston Scientific) in its mid-portion, just distal to the second diagonal branch. Total occlusion was confirmed by contrast injections after increasing balloon pressures in 1 atm increments. Total occlusion time was 60 minutes. Throughout the experiment, the animals were monitored by continuous digital ECG recordings and guide catheter pressure recordings. Digital cineangiograpic recordings were obtained in straight anterioposterior projections.

A total of 200 ml of contrast medium was administered to each animal. Approximately half of the contrast volume was used during diagnostic angiography and insertion of the guidewire into the LAD. The remaining contrast was injected after deflation of the balloon during the reperfusion period. After 20-30 minutes of reperfusion, catheter, balloon and guide wire were withdrawn. An Angio-Seal 6-French femoral artery closure device (St. Jude Medical, St. Paul, Minn.) was deployed and the Isoflurane was discontinued. After 30 minutes the ECG recording was stopped and the animals were extubated and transferred to the animal housing facility. No animal was lost during the housing period.

Twenty four hours later the animals were sedated and intubated. The thoracic cavity was opened by a midline sternotomy and ligatures were placed at the site of the proximal balloon occlusion, the aortic arch branches and the descending aorta. The right carotid artery was cannulated and a plastic catheter advanced into the aortic root. This cannula was then connected to a pressure transducer and 1.5 percent Evans Blue dye containing 100 mM potassium chloride. The vessels were occluded in the following order: LAD, aortic arch branches and descending aorta. The dye was then injected at a perfusion pressure of 120 mmHg until the myocardium was stained. The myocardium distal to the LAD ligature that did not stain was defined as the area at risk. In the NAC group two animals could not be used for the myocardial infarction analysis (insufficient Evans Blue staining, unsuccessful electrical cardioversion).

### Hemodynamic and Electrocardiographic Recording:

Blood pressure, heart rate, and high-resolution ECG were continuously recorded beginning at the time of coronary occlusion and for 30 minutes following reperfusion. The occurrence of ventricular tachycardia during coronary occlusion or reperfusion (defined as more than 4 consecutive ventricular ectopic beats) and ventricular fibrillation were recorded. Direct countercurrent cardioversion (DCCV) was provided for ventricular fibrillation and ventricular tachycardia with hemodynamic instability. No antiarrhythmic medications were used.

### Area at Risk and Infarct Measurements:

Evans Blue stained hearts were harvested and the atria were removed. The ventricles were sliced into one centimeter slices in a bread loaf fashion, with the first slice at the apex. At least five slices could be obtained in all hearts. The basal surfaces of the slices were sequentially imaged. Small samples of myocardium from areas of necrosis, area at risk and normal left ventricular myocardium were obtained from the apical side of the second and third slice and stored for additional analyses. Thereafter, all slices were incubated in a solution containing 1.5% (2,3,5)-triphenyltetrazolium chloride (FTC) in a phosphate buffered solution (pH 7.4, 37° C. for 25 min). The basal surfaces were then re-imaged. Area analysis (planimetry) was performed with the NIH-Image program (Scion Image Version 4.0.2). The size of the area at risk and infarct size for each slice was determined by multiplying the mass of the slice by the fraction of area at risk or by the fraction of area of infarction. Total infarct size and the area at risk size were expressed as a percentage of biventricular mass to account for the anterior right ventricular infarction.

### Infarct Morphology:

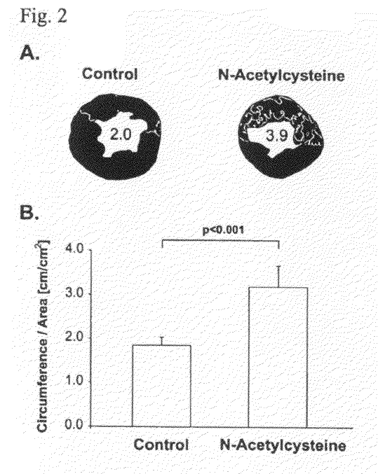

In order to quantify visual differences in infarct morphology, a circumference/area index of the infarcted myocardium using TTC stained sections was calculated. A low numerical value indicates a homogenous and discrete border zone, whereas a higher numerical value indicates a more complex, mottled border zone as demonstrated in FIG. 2.

### Apoptosis:

TUNEL assays were performed on samples from non-ischemic area at risk and infarcted tissue to investigate if NAC reduces ischemia-reperfusion induced apoptosis. A total of four samples from two control animals and six samples from three NAC treated animals were analyzed. Tissue sections were deparaffinized and permeabilized with proteinase K (25 μg/ml in 100 mM Tris.HCl). An in situ apoptotic cell death detection kit (TMR red; Roche Applied Bio Sciences, Indianapolis, Ind.) based on the TUNEL assay was used to detect apoptotic cells. Sections were mounted with Vectashield mounting medium containing 4′,6-diamidino-2-phenylindole (DAPI; Vector Laboratories, Burlingame, Calif.) as a nuclear counterstain. Sections were examined with a Zeiss Axiovert 200 microscope and Zeiss LSM 510 META laser scanning confocal microscope. The percentage of apoptotic nuclei per section was calculated by counting the total number of TUNEL-staining nuclei divided by the total number of DAPI-positive nuclei in 10 randomly selected fields at ×40 magnification (14).

### Serum Analysis

Serum was collected prior to contrast exposure and 24 hours after infarction and analyzed for creatinine. An attempt was also made to assay Cystatin C as a second renal marker. However, Cystatin C was undetectable in pig serum.

### Statistical Analysis

Endpoints were infarct and area at risk size, infarct morphology, percentage of TUNEL positive cells and change in serum creatinine at baseline and after 24 hours of reperfusion. Endpoints were compared between the contrast-NAC group and the contrast-only group using ANOVA analysis. In the dose escalation study the effect of each injection on blood pressure and QT interval was expressed as a percentage change from the immediately preceding baseline and analyzed by a paired student's t-test. A p-value of less than 0.05 was accepted as statistically significant. Results are reported and depicted as mean (±standard deviation).

### Results

Intracoronary NAC Dose Escalation: Blood Pressure and QT interval

Injections of contrast containing NAC did not cause any arrhythmias or changes in heart rate. Immediately after the injection of 5 ml contrast containing target dose NAC (11.3 mg/mL), a brief reduction of systolic blood pressures averaging 5 percent at the 6 seconds time point after the injection (p<0.05) was observed. This was accompanied by a transient 17 percent reduction in diastolic blood pressure (p<0.05). At 18 seconds, systolic pressure had returned to pre-injection pressures, whereas diastolic pressure continued to be decreased by about 7 percent (p<0.05). Complete normalization of blood pressure was achieved after 45 to 60 seconds. Control injections with contrast only did not show such effects.

During the dose escalation phase of the study, after a 250 mg bolus NAC injection systolic and diastolic blood pressure were reduced by 14 and 26 percent at six seconds (both, p<0.05) and 48 and 43 percent after a 500 mg PC-NAC bolus (both, p<0.05). After 18 seconds systolic blood pressure had normalized and diastolic pressures recovered after 45 to 60 seconds.

The use of a digital high resolution ECG recording system allowed detection of small variations in ECG time intervals. Six seconds after target dose injection the QT interval was to prolonged by 10 percent (p<0.05). At eighteen seconds after injection significant QT prolongation was no longer present. No significant QT changes were observed in animals receiving contrast injections. Immediately after the 250 mg and 500 mg NAC bolus injection, the QT interval was found to be prolonged by 16 and 35 percent respectively (both p<0.05). At 18 seconds, the QT interval prolongation normalized in the animals receiving 250 mg but was still prolonged by 9 percent with 500 mg injections (p<0.05). QT intervals in the latter group completely recovered to baseline values after 45 to 60 seconds.

### Effect of Intracoronary NAC on Myocardial Infarction

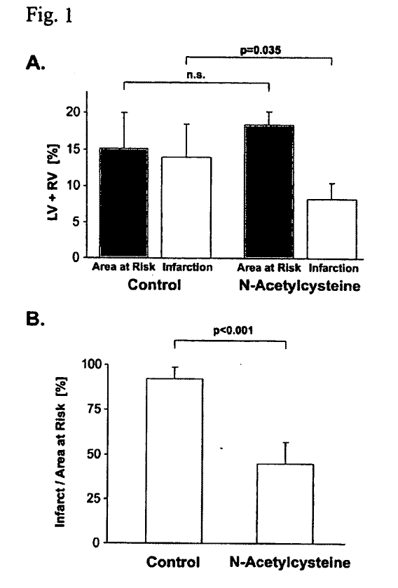

Comparison of the Non-Evans Blue stained areas reflecting the area at risk between NAC treated animals (n=5) and their controls (n=5) did not reveal a significant difference. Expressed as a percentage of ventricular myocardium, area at risk was 15.1±4.8% in control hearts and 18.4±1.8% in NAC treated hearts (p=0.2). Infarct size as percent of ventricular myocardium determined by TTC staining was 13.9±4.5 in controls and 8.2±2.3 in NAC treated animals (p<0.05). Infarct size expressed as a percentage of the area at risk was 92.4±6.3 percent in controls hearts and 44.7±12.4 percent in NAC treated hearts (p<0.01), suggesting a NAC-mediated protection from infarct progression (FIG. 1).

The morphologic appearance of the infarcted area differed markedly between the two groups. Infarcts in control animals were homogeneous with discrete borders while infarcts in NAC treated animals contained areas of viable myocardium interspersed with infarcted areas resulting in a mottled appearance. Reflecting this difference, the ratio of infarct circumference to infarct area was greater in NAC treated animals (FIGS. 2A and 2B).

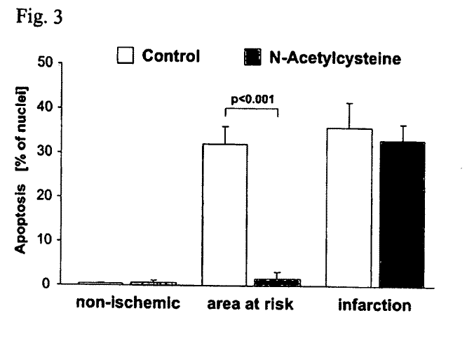

To investigate if the protective effect of NAC is mediated by protection from apoptosis TUNEL assays were performed. In myocardium obtained from non-LAD territories (non-ischemic) we found a low number of TUNEL positive myocyte nuclei without a significant difference between NAC treated and control animals. Within the infarct tissue of control animals 35.8±5.7 percent of nuclei stained positive for apoptosis compared to 33.0±3.7 percent in NAC treated animals (p=0.4). In contrast, in tissue obtained from the non-infarcted area at risk obtained from control animals 32.1±4.0 percent of nuclei stained TUNEL positive, whereas this value was 1.6±1.6 percent in NAC treated animals (p<0.001), suggesting NAC mediated protection from apoptosis (FIG. 3).

### Periprocedural Arrhythmias

Neither animal in the dose escalation study and none of the control ischemia-reperfusion animals developed ventricular fibrillation or required DCCV. One control animal had a brief episode of self terminating ventricular tachycardia during reperfusion. In contrast, all NAC treated animals developed ventricular tachycardia, fibrillation or both during ischemia-reperfusion. All of these events occurred either in the second half of the occlusion or during the first 15 minutes of reperfusion. All animals underwent successful DCCV with the exception of one in whom cardioversion was delayed by about four minutes. One animal in the NAC group that could not be used for the morphometric MI analysis due to insufficient Evans Blue staining had an episode of ventricular tachycardia followed by fibrillation after initiating anesthesia on day two of the study. Periprocedural arrhythmias are summarized in Table 1.

TABLE 1
Arrhythmia during Ischemia-Reperfusions

| | Ischemia | | | Reperfusion | | |
|---|---|---|---|---|---|---|
| | VT | VF | DCCV | VT | VF | DCCV |
| C-1 | − | − | − | − | − | − |
| C-2 | − | − | − | − | − | − |
| C-3 | − | − | − | − | − | − |
| C-4 | − | − | − | + | − | − |
| C-5 | − | − | − | − | − | − |
| N-1 | − | − | − | + | + | + |
| N-2 | + | − | − | − | − | − |
| N-3 | − | − | − | + | − | − |
| N-4 | − | − | − | − | + | + |
| N-5 | + | + | +* | − | − | − |
| N-6 | − | − | − | + | + | + |
| N-7 | + | + | + | − | − | − |

Ventricular tachycardia (VT), ventricular fibrillation (VF) and direct countercurrent cardioversion (DCCV) in pigs undergoing an ischemia-reperfusion protocol. The animals C-1 to C-5 received Iopamidol contrast-only, whereas the animals N-1 to N-7 received N-Acetylcysteine added to the Iopamidol contrast agent.
*animal died after delayed DCCV.

### Effect of Intracoronary NAC on Serum Creatinine Levels

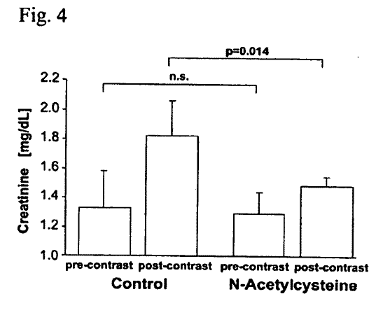

Blood samples for creatinine were obtained prior to the administration of the contrast agent via the femoral sheath and then 24 hours after infarction from the aortic root cannula. Baseline creatinine level prior to contrast medium administration was 1.32±0.26 mg/dL in the control group and 1.29±0.15 mg/dL in the NAC treated group. Twenty four hours after ischemia-reperfusion creatinine levels rose to 1.82±0.24 mg/dL in the control group and 1.49±0.06 mg/dL in the NAC treated animals (p<0.05). These data are summarized in FIG. 4.

### Discussion

The present study demonstrates that intra-coronary administration of a radiographic contrast agent mixed with N-Acetylcysteine at doses similar to those used in a recent clinical trial provides cardiorenal protection in a model of ischemia-reperfusion. Infarct size as a percentage of area at risk was reduced by about half and the rise in serum creatinine following contrast administration was blunted in NAC-treated animals.

### Direct Effects of Intra-Coronary NAC.

A small transient QT prolonging effect of NAC at the target dose of 11.3 mg/mL contrast was observed after intracoronary bolus injections. The transient, mild decrease in both systolic and diastolic blood pressure may indicate a myocardial depressant or a vasodilator effect. However the latter appears more likely in light of a previous human trial of intracoronary NAC administration (480 milligrams over 10 minutes) that found a NAC induced improvement of endothelial function (11). It has been reported that NAC may enhance the bioavailability of NO by forming biologically active byproducts or by scavenging the oxygen free radicals that are largely responsible for the short half life of NO (15). The blood pressure effect in healthy pigs was small and transient in nature and therefore unlikely to be of physiologic significance.

### Cardiac and Renal Protection.

The antioxidative properties of NAC have been implicated as the mechanism of its cardioprotective effect (5). In a canine model of myocardial ischemia-reperfusion both infarct size and reperfusion arrhythmias were reduced when intravenous NAC was given mostly during reperfusion (16). In two small clinical studies, intravenous NAC was combined with fibrinolytic therapy in patients with acute ST elevation myocardial infarction (5, 6). A decrease in infarct size, improved R-wave recovery were reported in association with improved global and regional ejection fraction. The present study differs from the previous animal and human studies in that NAC was administered both before and after reperfusion and via the intra-coronary route. The data presented herein indicate that a component of the approximately 50 percent reduction in infarct size is mediated by NAC induced protection from apoptosis that may slow the progression of the infarction. This effect could be explained by NAC-induced preconditioning or reduction in reperfusion injury (17).

Interestingly, the infarct morphology in animals treated with the contrast-NAC mixture differed from the contrast-only animals. Analysis of the infarcted myocardium revealed viable areas within the infarct zone in the NAC treated animals, resulting in a heterogenous and mottled appearance; on the other hand, infarcts in control animals were homogenous, with distinct border zones.

The 37 percent increase in creatinine levels in healthy young pigs with an average body weight of 28 kilograms was not unexpected considering that a total of 200 ml of contrast was administered without periprocedural hydration. This observation underscored the impact of contrast volume on renal function. The results of the studies described herein indicate that NAC provided during contrast administration reduced the rise in post procedure creatinine levels by about 60 percent. This result may indicate that pre-procedural administration of NAC is not a prerequisite for its renal protective effect. The exact mechanism whereby NAC mediates renal protection remains to be elucidated. However, the most likely mechanism is its antioxidant properties (18).

### Arrhythmias.

The results presented herein indicate that NAC at a concentration of 11.3 mg/mL could be safely delivered as a bolus to the coronary circulation during diagnostic angiography in exactly the same fashion as contrast agents without inducing hemodynamic instability, arrhythmias or lasting ECG changes. In contrast, it was found an increase in ventricular tachycardia and fibrillation during ischemia-reperfusion in animals that received NAC. It is worth emphasizing that no animal died during the 24 hour period of housing that followed the procedure and none of the arrhythmias occurred with or immediately after injection of the NAC-contrast mixture.

The resulting ventricular arrhythmias were unexpected. It contrasts with a study in which NAC administration at the time of reperfusion in a canine model was associated with a marked reduction in ventricular arrhythmias (16). However, it is well documented that areas of viable myocardium interspersed within infarcted areas provide foci and re-entry pathways for ventricular arrhythmias (19, 20). It was possible to successfully cardiovert all animals with the exception of one in whom cardioversion was delayed. Nonetheless, this observation is concerning with respect to the potential clinical use of intra-coronary NAC. Additional studies will be required to assess the occurrence and possible prevention of these arrhythmias in a setting that more closely mimics primary coronary intervention in patients with acute MI where a NAC contrast mixture would be administered exclusively with reperfusion.

In conclusion., these studies now have demonstrated the feasibility and efficacy of delivering NAC in conjunction with an intracoronary contrast agent to provide combined cardiac and renal protection in the setting of ischemia-reperfusion. This observation may have potential application in clinic practice. The use of contrast agents enriched with NAC would guarantee that patients who receive large amounts of contrast medium and are therefore at a higher risk of CIN would be protected by larger doses of NAC. Furthermore, these results are consistent with prior studies suggesting that NAC provides significant myocardial protection during ischemia-reperfusion. As noted earlier, the potential risk of ventricular arrhythmias as well as the magnitude of cardiac and renal protection need to be carefully assessed in future animal studies that more closely simulate clinical practice in the setting of acute myocardial infarctions.

### REFERENCES

(1) Rihal C S, et al. Circulation. 2002; 105:2259-2264.
(2) Mehran R. & Nikolsky E. Kidney Int Suppl. 2006; 100:S11-S15.
(3) Mehran R. et al. J Am Coll Cardiol. 2004; 44:1393-1399.
(4) Barrett B J & Parfey P S. N Engl J Med 2006; 354: 379-386.
(5) Arstall M A. et al., Circulation. 1995; 92:2855-2862.
(6) Sochman J. et al., Clin Cardiol. 1996; 19:94-100.
(7) Tepel M. et al., N Engl J Med 2000; 343:180-184.
(8) Birck R, et al., Lancet. 2003; 362:598-603.
(9) Alonso A, et al., American Journal of Kidney Diseases. 2004; 43:1-9.
(10) Marenzi G, et al., N Engl J Med. 2006; 354:2773-2782.
(11) Andrews N P. et al., J Am Coll Cardiol. 2001; 37:117-123.
(12) Vale J A. & Proudfoot A T. Lancet 1995; 346:547-52.
(13) Meyer M, et al., J Interv Cardiol. 2006; 19:401-404.
(14) Singla D K et al., Am J Physiol Heart Circ Physiol. 2007; 293:H1308-1314.
(15) Scharfstein J S et al., J Clin Invest. 1994; 94:1432-1439.
(16) Sochman J et al., Int J Cardiol 1990; 28:191-196.
(17) Singla D K et al., Can J Physiol Pharmacol. 2007; 85:439-443.
(18) Drager L F et al., Nephrol Dial Transplant. 2004; 19:1803-1807.
(19) de Bakker J M, et al., Circulation. 1988; 77:589-606.
(20) Schmidt A et al., Circulation. 2007; 115:2006-2014.

### EQUIVALENTS

Those skilled in the art will recognize, or be able to ascertain using no more than routine experimentation, many equivalents to the specific embodiments of the invention described herein. Such equivalents are intended to be encompassed by the following claims.

All references disclosed herein, including patent documents, are incorporated by reference in their entirety.

We claim:

1. A composition comprising a contrast agent and N-acetylcysteine (NAC).

2. The composition of claim 1, wherein the NAC is crosslinked to the contrast agent.

3. The composition of claim 1, wherein the amount of NAC in the composition is about 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, or more of NAC per mL (cc) of contrast agent.

4-7. (canceled)

8. A method for administering a contrast agent to a subject, the method comprising:
administering the composition of claim 1 to the subject, wherein the contrast agent and N-acetylcysteine (NAC) of the composition are simultaneously administered to the subject.

9. The method of claim 8, wherein the NAC is crosslinked to the contrast agent.

10. The method of claim 8, wherein the amount of NAC administered in the composition is an amount effective to prevent organ damage in the subject.

11. The method of claim 10, wherein the organ damage is myocardial ischemia, myocardial infarction, and/or reperfusion damage.

12-13. (canceled)

14. The method of claim 10, wherein the organ damage is contrast-induced nephropathy (CIN).

15-17. (canceled)

18. The method of claim 8, wherein the subject is a mammal.

19-24. (canceled)

25. A composition comprising a contrast agent and an antioxidant compound.

26. The composition of claim 25, wherein the antioxidant compound is N-acetylcysteine (NAC).

27. The composition of claim 25, wherein the antioxidant compound is crosslinked to the contrast agent.

28. The composition of claim 25, wherein the antioxidant compound is sodium thiosulfate (STS), GSH ethyl ester, D-methionine, thiol amifostine (Ethyol, WR2721), Vitamin E, tocopherol, dithiotreitol, mercaptoethanol, or glutathion.

29. The composition of claim 25, wherein the amount of the antioxidant compound in the composition is from about 0.1 mg to at least about 200 mg or more of antioxidant per mL (cc) of contrast agent.

30-34. (canceled)

35. A method for administering a contrast agent to a subject, the method comprising: administering the composition of claim 25 to the subject, wherein the contrast agent and the antioxidant compound of the composition are simultaneously administered to the subject.

36. The method of claim 35, wherein the antioxidant compound is crosslinked to the contrast agent.

37. The method of claim 35, wherein the amount of antioxidant compound administered in the composition is an amount effective to prevent organ damage in the subject.

38-44. (canceled)

45. The method of claim 35, wherein the subject is a mammal.

46-48. (canceled)

49. The method of claim 35, wherein the contrast agent is an ionic contrast agent.

50. (canceled)

51. The method of claim 35, wherein the amount of antioxidant compound administered to the subject is from about 0.1 mg to 200 mg or more of antioxidant compound per mL (cc) of contrast agent administered to the subject.

52. The method of claim 35, wherein the antioxidant compound is NAC.

53. (canceled)

54. A kit comprising a contrast agent and N-acetylcysteine (NAC), and optionally comprising directions for crosslinking the NAC to the contrast agent and/or directions for administering the contrast agent and the NAC to a subject.

55-85. (canceled)
